(19) United States
(12) Patent Application Publication
(10) Pub. No.: US 2020/0405572 A1
(43) Pub. Date: Dec. 31, 2020

(54) COLPOSUCTION SYSTEM AND METHODS FOR AUGMENTATION AND REJUVENATION OF VAGINAL TISSUE

(71) Applicant: Allan Yang Wu, Rancho Mirage, CA (US)

(72) Inventor: Allan Yang Wu, Rancho Mirage, CA (US)

(21) Appl. No.: 16/457,584

(22) Filed: Jun. 28, 2019

(51) Int. Cl. A61H 19/00; A61H 9/00

(52) U.S. Cl. CPC A61H 19/40; A61H 9/0057; A61F 2/0059; A61H 2201/169; A61H 2201/0188; A61H 2201/1645

(57) ABSTRACT

Colposuction systems for applying negative pressure against walls of a vaginal canal, which includes an elongated body with an inner chamber that slidably houses a piston block, a plurality of channels that fluidly couple the inner chamber to an outer surface of the body; and means for pulling the piston block proximally thereby applying negative pressure through the plurality of channels. Methods for inducing beneficial effects in vaginal tissue which include applying negative pressure to vaginal tissue along a length of the vaginal wall using the colposuction device to cause edema and/or hypertrophy of the vaginal tissue.

### FIELD OF THE INVENTION

The invention relates to colposuction systems and methods for augmentation and rejuvenation of vaginal tissue.

### BACKGROUND OF THE INVENTION

Vaginal rejuvenation is a broad term that covers several vaginal corrective procedures. These procedures are primarily meant to correct problems that can occur after giving birth or during the aging process. These conditions can include laxity (lack of tightness) of the vagina, urinary incontinence, and lack of lubrication. The two main groups of women seeking vaginal rejuvenation include postmenopausal women and postpartum women.

A variety of treatments are currently available for vaginal rejuvenation. Among these include kegel or pelvic floor exercises, which are meant to strengthen the muscles in and around the vagina; vaginoplasty, which is a surgical procedure that tightens the vagina; and energy-based treatments. The two energy-based treatments commonly used are radiofrequency (RF) treatment and laser treatment. An example of radiofrequency (RF) treatment is described in US 2018/0303542 by Eclipse Aesthetics, LLC, which seeks to increase local production of collagen by heating the walls of the vaginal cavity. An exemplary laser treatment is described in US 2017/0296272 by Alma Lasers, Ltd, where a laminated laser system delivers mid-IR wavelengths to the upper layers of the vaginal tissue, and like RF procedures, also causing the lower layers to produce more collagen.

Dermal fillers have also been used in vaginal tissue for purposes of tightening and sexual enhancement (including improved stimulation of the G-spot during intercourse). Dermal filling procedures typically involve injecting a hyaluronic acid-based composition into the front wall of the vagina, which works to fill out the area by absorbing water, which in turn, improves sensitivity. Adipose tissue has also been proposed for use as a dermal filler.

Since the above procedures are generally considered cosmetic in nature, they are typically not covered by insurance policies, which shifts the financial burden to the subject desiring such treatments. In addition, since the procedures are typically performed by a physician, they can be cost prohibitive for many. Accordingly, there remains a need for alternative approaches for vaginal rejuvenation.

Negative pressure therapy against the surface of non-muscosal, wounded skin has been used clinically for wound healing. The underlying technical approach of the therapy is to apply negative pressure to a local region, which in turn, draws nutrients and reparative cells to the site of injury, which ultimately accelerates the wound repair process. Negative pressure therapy has also been applied against non-muscosal skin for tissue augmentation procedures. For example, U.S. Pat. No. 5,536,233 discloses a system, which includes a dome-shaped apparatus configured to apply negative pressure to the breasts, which causes expansion and hypertrophy, thereby allowing maximal transfer of autologous fat for the purpose of breast augmentation.

Relatedly, US 2016/0242953 by Bridges proposes a suction-based device for increasing blood flow to the vaginal walls and inducing muscle contractions, which results in tightening. Broadly, the device includes a shaft with suction ports connected to a handpump (or air powered suction device) and vented slots for inducing a vacuum. While the approach of Bridges suggests the use of a suction-based device for increasing blood flow to vaginal tissue and increasing tone of vaginal muscle, it does not disclose use for tissue regeneration or repair, nor for other cosmetic procedures.

### SUMMARY OF THE INVENTION

The invention provides systems and methods that apply negative pressure to the vaginal walls of a subject for regeneration and/or repair of vaginal tissue. By applying low to moderate levels of negative pressure, blood circulation into the vaginal tissue can be increased for improved sexual sensation; whereas, applying higher levels of negative pressure to vaginal tissue can cause localized micro-traumas, which stimulate tissue repair and regeneration by tissue repair nutrients and reparative cells recruited from the blood stream during hypertrophy and edema. Further, the systems provide an effective pretreatment prior to vaginal dermal filling and vaginal fat filling procedures.

The above is accomplished by way of a colposuction system for applying negative pressure against walls of a vaginal canal, which includes an elongated body having an inner chamber that slidably houses a piston block, the piston block being sealed air-tight against the inner chamber. A plurality of channels, which are positioned distal to the piston block, fluidly couple the inner chamber to an outer surface of the body. The colposuction system is activated by a means for pulling the piston block proximally, thereby applying negative pressure through the plurality of channels and against the vaginal tissue.

In some embodiments, the body can include a rigid core surrounded by a deformable or compressible coating layer. If the rigid core is made hollow, its lumen can also be used as the inner chamber for the piston block. Exemplary means for pulling the piston block proximally include an automated pump, a squeeze bulb pump, and a pull line coupled to the piston block.

In preferred embodiments the body can be deformed (e.g. compressed) for contacting and following the contour of the walls of the vaginal canal when sufficient negative pressure is applied. For example, in some embodiments, the body is configured to deform so that at least 50% of a body surface area, defined by a region encompassing the plurality of channels, contacts vaginal tissue when the negative pressure is applied. In further embodiments, the body is configured to deform so that at least 75% of the body surface area, defined by the region encompassing the plurality of channels, contacts vaginal tissue when the negative pressure is applied. In still further embodiments, between 75% and up to 100% of the body surface area, defined by a region encompassing the plurality of channels, contacts vaginal tissue when the negative pressure is applied.

The invention also encompasses use of the colposuction system as a pretreatment for vaginal dermal filling or vaginal filling with fat. Exemplary methods of pretreating vaginal tissue for vaginal dermal filling or vaginal filling with fat, include providing a patient for a vaginal dermal filling or a vaginal filling with fat procedure; inserting the body of the culposuction system into the vagina of the patient; applying negative pressure within the chamber so that the body contacts the vaginal tissue along a length of the vaginal wall that is to be treated with the dermal filling or fat filling procedure; maintaining sufficient negative pressure over time or repeatedly applying sufficient negative pressure to cause edema of the vaginal tissue along the length of the vaginal wall to be treated; releasing the negative pressure and removing the body from the vagina; and designating the swollen length of vaginal tissue as a pretreated region for the vaginal dermal filling or vaginal fat filling procedure while the pretreated region remains swollen. In some embodiments the maintained negative pressure or repeatedly applied negative pressure is from 1 mmHg to 180 mmHg. The invention also encompasses treatment where the vaginal dermal filling or vaginal filling with fat is subsequently performed on vaginal tissue.

The invention also encompasses use of the colposuction system for inducing vaginal tissue regeneration. Exemplary methods of inducing vaginal tissue regeneration include providing a patient in need of vaginal tissue regeneration; inserting the body of the colposuction system into the vagina of the patient; applying negative pressure within the chamber so that the body contacts the vaginal tissue along a length of the vaginal wall to be regenerated; maintaining sufficient negative pressure over a sufficient time or repeatedly applying sufficient negative pressure to cause micro-trauma of the vaginal tissue along the length of the vaginal wall to be regenerated; and releasing the negative pressure and removing the body from the vagina. Exemplary ranges for negative pressure to induce micro-trauma include 1 mmHg to 180 mmHg.

The invention also encompasses use of the colposuction system for inducing edema and/or hypertrophy of vaginal tissue. Exemplary methods of causing or inducing edema and/or hypertrophy of vaginal tissue include applying a sufficient amount of negative pressure to a subject's vaginal tissue with the colposuction system and for a sufficient time to cause edema and/or hypertrophy of the vaginal tissue.

In a specific application of the above, the invention provides use of the colposuction system for improving sexual sensation in a subject or partner. Exemplary methods of improving sexual sensation in a subject or a partner include providing a subject in need of improving sexual sensation or having a partner in need of improving sexual sensation; and performing the method of causing or inducing edema and/or hypertrophy of vaginal tissue on the subject. In a further use of the colposuction system, the negative pressure is applied to an area designated as a G-spot in the subject.

The invention also encompasses use of the colposuction system for affecting vaginal muscle tissue in a subject. Exemplary methods for affecting vaginal muscle tissue in a subject include applying a sufficient amount of negative pressure to a subject's vaginal walls with the colposuction system and for a sufficient time to cause edema around the vaginal muscle tissue; and periodically repeating the application. However, significantly better results can be achieved if the amount of negative pressure causes micro-trauma at the muscle. In particular a negative pressure of 1 mmHg-180 mmHg is preferred.

In a specific application, the invention provides use of the colposuction system for treating or ameliorating urinary incontinence in a subject. Exemplary methods of treating or ameliorating urinary incontinence in a subject include providing a subject suffering from urinary incontinence; and performing the method of affecting vaginal muscle tissue in the subject until urinary incontinence is reduced or prevented. Other exemplary methods of treating or ameliorating urinary incontinence in a subject include pretreating vaginal tissue for vaginal dermal filling or vaginal filling with fat on the subject; and performing a dermal filling or fat filling procedure on the pretreated tissue.

In another specific application, the invention provides use of the colposuction system for preventing or ameliorating pelvic organ prolapse in a subject. Exemplary methods of preventing or ameliorating pelvic organ prolapse in a subject include providing a subject suffering from or at risk of suffering from pelvic organ prolapse; and periodically performing the method of affecting vaginal muscle tissue in a subject. Other exemplary methods of preventing or ameliorating pelvic organ prolapse in a subject include pretreating vaginal tissue for a vaginal dermal filling or vaginal filling with fat procedure on the subject; and performing a dermal filling or fat filling procedure on the pretreated tissue.

In another specific application, the invention provides use of the colposuction system for progressive dilation of a stenoted vaginal canal. Exemplary methods for progressive dilation of a stenoted vaginal canal include providing a subject suffering from a stenoted vaginal canal; and periodically performing the method of affecting vaginal muscle tissue in a subject until the canal dilates.

### BRIEF DESCRIPTION OF THE DRAWINGS

FIG. 1 depicts an exemplary system for applying negative pressure to the vaginal canal.

FIG. 2 depicts another exemplary system for applying negative pressure to the vaginal canal.

### DETAILED DESCRIPTION OF PREFERRED EMBODIMENTS

In accordance with various embodiments herein, a colposuction system is provided that delivers a therapeutically effective, negative force to vaginal tissue, which induces tissue repair and/or regeneration and/or rejuvenation. More specifically, use of the colposuction system induces micro-traumas, namely, small tears or abrasions, to the vaginal tissue and because of the simultaneous influx of reparative cells and nutrients during tissue edema, vaginal tissue is repaired, regenerated, and/or rejuvenated. Short term beneficial effects include increased sexual sensation; whereas, long term effects provide longer term sexual satisfaction, vaginal muscle toning, and vaginal tissue repair and regeneration. Moreover, the colposuction system also improves the effectiveness of dermal filling procedures by permitting the injection of higher loads of fillers.

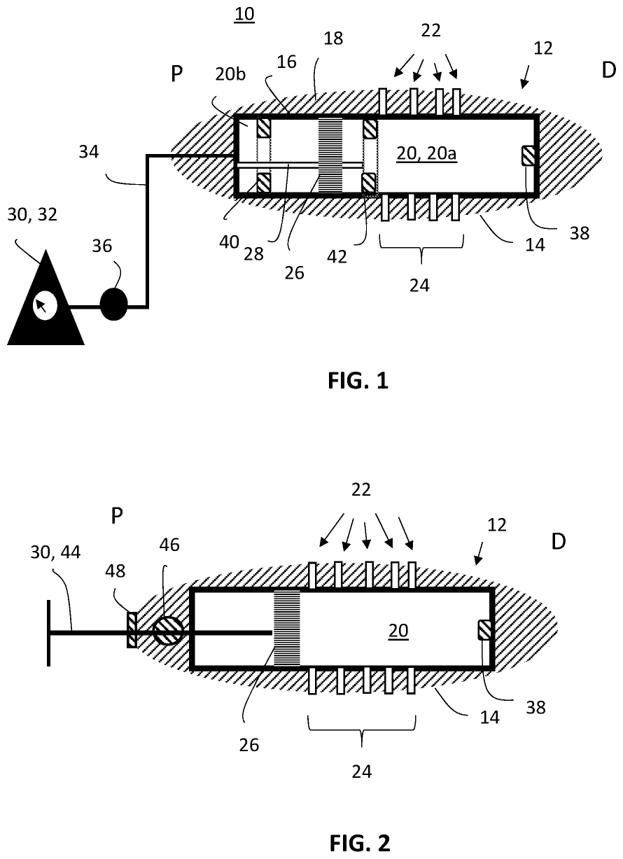

Referring now to the drawings, which are shown for purposes of illustrating embodiments of the invention only and not for purposes of limiting the same, FIGS. 1 and 2 depict exemplary colposuction systems 10 as a basis for carrying out methods described herein. Beginning at FIG. 1, an exemplary colposuction system 10 includes an elongated body 12, which can be described with reference to its proximal P end and distal D ends. The body 12 extends longitudinally along a longitudinal axis and is sized and configured for insertion into a patient's vagina. Preferably, when inserted into the vagina for treatment, at least a portion of the body 12 forms an annular seal against the walls of the vagina, and as will be described in more detail, as negative pressure is applied, the surface 14 of the body 12 increasingly contacts vaginal tissue. That is, as negative pressure is applied through the system 10 a greater portion of the body 12 contacts vaginal tissue. In some embodiments, this is cause by pulling the vaginal tissue inward and against the body 12; whereas in other embodiments, this is cause by pulling the body 12 outward and against the tissue. In some embodiments, the body 12 has a profile so that when inserted into the patient's vagina, at least 20% of its inserted length fits against walls of the vaginal canal during use. In further embodiments, at least 30% of its inserted length fits against walls of the vaginal canal during use. In further embodiments, at least 40% of its inserted length fits against walls of the vaginal canal during use. In further embodiments, at least 50% of its inserted length fits against walls of the vaginal canal during use. In further embodiments, at least 60% of its inserted length fits against walls of the vaginal canal during use. In further embodiments, at least 70% of its inserted length fits against walls of the vaginal canal during use. In further embodiments, at least 80% of its inserted length fits against walls of the vaginal canal during use. In further embodiments, at least 90% of its inserted length fits against walls of the vaginal canal during use. In further embodiments, up to 100% of its inserted length fits against walls of the vaginal canal during use. The term “inserted length” refers to the portion of the body 12 that begins at the annular seal with the vagina and proceeds distally D. Forming the profile for fitting against the walls of the vaginal wall can be accomplished by manufacturing the body 12 from materials that are sufficiently compressible and/or bendable so they are able to compress and/or bend to follow the contours of the walls of the vaginal canal. Exemplary materials for manufacturing the body 12 include silicone, rubber, bendable and/or compressible polymers, polyethylene, polytetrafluoroethylene (PTFE) and others. In some embodiments, the body 12 is made from a single material having a uniform density and/or rigidity. In other embodiments, the body 12 is formed from two or more different materials such that the rigidity and/or density differs in two or more portions of the body 12. In some embodiments, a rigid hollow core 16 is formed from a rigid polymer that is not easily deformable, which is itself surrounded by a layer 18 of material that is deformable under pressures typical associated with vaginal forces against the body 12 at 1 mm to 180 mmHg.

Within the body 12 is an inner chamber 20 that is fluidly coupled to the outer surface 14 by way of a plurality of channels 22, preferably distal D to and/or at the site of the annular seal after insertion. As such, modulating the atmospheric pressure within the chamber 20 affects the atmospheric pressure within the vagina via the channels 22. Further, when used to induce micro-traumas along vaginal tissue, such micro-traumas are formed primarily at regions where the channels 22 contact the vaginal wall. In some embodiments, abrasions or small tears of vaginal tissue occur by compressing the vaginal tissue against rigid channels 22. As such, deformation of the body surface 14 may be more critical at regions defined generally by the presence of channels 22. For example, in some embodiments the body 12 is configured to deform during use so that at least 50% of a body surface area 24, defined by a region encompassing the plurality of channels 22, contacts vaginal tissue when the negative pressure is applied. In further embodiments, the body 12 is configured to deform so that at least 75% of a body surface area 24, defined by a region encompassing the plurality of channels 22, contacts vaginal tissue when the negative pressure is applied. In still further embodiments, from 75% to 100% of the body surface area 24, defined by a region encompassing the plurality of channels 22, contacts vaginal tissue when the negative pressure is applied.

As used herein “a body surface area, defined by a region encompassing the plurality of channels” refers to a contiguous area along the outer surface 14 of the body 12 that includes a group of at least five channels 22 and can include up to all channels 22. The outer boundary of the contiguous area is defined by the outermost channels 22 within the group.

The skilled artisan will now appreciate that the plurality of channels 22 can be arranged in any suitable pattern and can have a same or different pattern over the body 12, which can be one or more of a symmetrical pattern, a non-symmetrical pattern, a random pattern, and/or any other suitable pattern. The skilled artisan will now appreciate that depending on the needs of the user, the plurality of channels 22 may be located on only one portion or side of the body 12 or can be on multiple portions and/or sides of the body 12. However, as general guidance, the channels 22 are typically positioned more distal D than proximal P and are preferably positioned distal D to the piston block 26. In some embodiments, one or more channels 22 are positioned for localized contact with a G-spot. In some embodiments, the channels 22 are formed by the user after initial assessment of the vaginal walls so that the channels 22 can selectively arrayed for contact against a region of interest along the vaginal wall. Likewise, the size and shape of the channel 22 opening can vary depending on the needs of the user. For example, fewer, larger channels 22 may be preferred for targeting greater micro-trauma such as for tissue regeneration or for preparing regions for dermal filling and/or fat filling; whereas, smaller channels 22 spread over the majority of the body 12 may be preferred for primarily increasing blood flow throughout the vaginal walls.

Pressure modulation is performed by way of a piston block 26, which is housed within and forms an air-tight seal around its perimeter with the inner chamber 20. The piston block 26 is configured to slide proximally P and distally D along the longitudinal axis, optionally, along a sliding track 28. Drawing the piston block 26 proximally along the longitudinal axis decreases pressure in the distal portion 20a of chamber 20, which pulls air inward through the plurality of channels 22 and because of the annular seal, pulls the body 12 and the walls of the patient's vagina against each other. Likewise, releasing the forces that cause the drawing of the piston block 26 proximally, permit the piston block 26 to slide distally D, thereby equalizing pressure within the inner chamber 20 to ambient air and thus releasing suction forces against the walls of the vagina for its release.

As shown in FIG. 1, the means for pulling 30 the piston block 26 proximally P can be an automated pump 32. In particular, pump 32 with integral pressure regulator is shown communicatively coupled to proximal chamber portion 20b way of tubing 34 connected through a valve 36 having three positions, namely, “off”, which prevents communication through the tubing 34; “on”, which fluidly connects the pump 32 to inner chamber 20, thereby permitting the pump 32 to reduce the pressure from the proximal chamber portion 20b and move the piston block 26 proximally P; and “purge”, which depressurizes tubing 34 to the ambient air, thereby releasing pressure from the proximal chamber portion 20b to cause the piston block 26 to move distally D. While means for pulling 30 the piston block 26 proximally P shown in FIG. 1 is an automated pump 32 with electronic controller configured to adjust pressure to a predefined or a user defined parameter (e.g. programmed or programmable pressure), a manual pump, such as a squeeze bulb could also be used. When the means for pulling 30 the piston block 26 proximally P is a squeeze bulb, a pressure regulator can be provided to regulate pressure within the tubing 34. The skilled artisan will appreciate that the inner chamber 20 can also be equipped with one or more pressure sensors 38 to monitor the pressure within the distal portion 20a of the inner chamber 20.

Optionally, a proximal stop 40 is provided within the inner chamber to further limit pressure differences within the system 10. Optional proximal stop 40 and distal stop 42 are shown in FIG. 1. Proximal stop 40 limits movement of the piston block 26 proximally P and distal stop 42 limits movement of the piston block 26 distally D, such as to ensure the piston block 26 remains proximate P to the channels 22. By providing stops 40, 42 the pressure of the distal portion 20a of the inner chamber 20 can be further regulated by effectively providing a safety valve within the chamber 20 that limits the pressure difference across the piston block 26 by providing an air-tight sealing when piston block 26 is pressed against proximal stop 40 or distal stop 42.

Turning now to FIG. 2, another exemplary means for pulling 30 the piston block 26 proximally is provided, which includes a pull line 44 (e.g. rod, strap or string) coupled to the piston block 26. Pulling the pull line 44 draws the piston block 26 proximally P along the longitudinal axis, which decreases pressure in the chamber 20, thereby sucking air inward through the plurality of channels 22 and because of the annular seal, pulls the body 12 and the walls of the patient's vagina against each other. A pressure sensor 38 positioned within the inner chamber 20 can provide instant feedback regarding the pressure within the inner chamber 20. Alternatively, a scale 46 can be functionally coupled to the body 12 to provide a readout of internal pressure based upon the pulling force on the pull line 44. Once applied, pressure can be held constant by way of a locking mechanism 48, such as a slide lock that locks the pull line 44 in place and thus the piston block 26.

Referring collectively to FIGS. 1 and 2, though nonlimiting, the means for pulling 30 the piston block 26 proximally P can be configured to reduce atmospheric pressure within the inner chamber 20 from 760 mmHg to less than 180 mmHg. In some embodiments, pressure within the chamber 20 is reduced to less than 150 mmHg. In some embodiments, pressure within the chamber 20 is reduced to less than 125 mmHg. In some embodiments, pressure within the chamber 20 is reduced to less than 100 mmHg. In some embodiments, pressure within the chamber 20 is reduced to less than 75 mmHg. In some embodiments, pressure within the chamber 20 is reduced to less than 50 mmHg. In some embodiments, pressure within the chamber 20 is reduced to less than 25 mmHg. In some embodiments, pressure within the chamber 20 is reduced to not less than 1 mmHg. The skilled artisan will appreciate that while high pressures (e.g. between 180-600 mmHg) can be used to increase blood flow, lower pressures (e.g. 1-180 mmHg) can be used to induce microtrauma for tissue regeneration or provide pretreatment for subsequent dermal filling procedures. As such, the applied pressure can vary depending on the intended result.

With regards to both FIGS. 1 and 2, releasing the force(s) that causes drawing the piston block 26 proximally P permits the piston block 26 to slide distally D through the inner chamber 20, thereby releasing suction forces from the walls of the vagina for release of the body 12 for or repeated use of the system 10 in a same or different procedure.

As should be apparent, the disclosure is not intended to limit the system to the precise forms or particular fields of use disclosed. Rather, it is contemplated that various alternate embodiments and/or modifications to the system 10, whether explicitly described or implied herein, are possible for the below described methods of use. For example, it is contemplated that the various embodiments set forth herein may be combined together and/or separated into additional embodiments where appropriate.

As non-limiting examples of modifications, the system 10 can also include a vibrating feature so as to distract the patient from any discomfort caused by the changing pressures against vaginal tissues. The vibrating feature may also distract the patient from any discomfort caused by the insertion of the body 12 into the vaginal cavity. In order to include a vibrating feature, the system 10 may include a vibration-generating motor, which may be connected to a programmable controller and a power source. The vibration-generating motor may be automated with certain properties, such as different vibration frequencies, in order to provide patient comfort or distraction from any discomfort caused by changing pressures against the vaginal wall. One of ordinary skill in the art will appreciate the construction, choice of type of the vibration-generating motor, programming, and operation. Functionality of such vibration-generating motor is well known, rendering further description of such devices unnecessary in this regard.

As another non-limiting example, the system 10 can include embedded light emitting diode (LED) lighting so as to assist with insertion and positioning of the body. LEDs use heat sinks to absorb the heat produced by the LED, and thus prevent injury of vaginal tissue by overheating.

In furtherance of the above description of the colposuction system 10, the invention also includes use of the colposuction system 10 to achieve one or more beneficial effects, such as, but not limited to, improved sexual sensation to the subject and/or the subject's partner, a pretreatment prior to dermal filling or dermal filling with fat, treatment of vaginal atrophy and dryness, treatment of urinary incontinence and pelvic organ prolapse, treatment of vaginal canal stenosis, G-spot localization, micro-trauma introduction for the purpose of tissue regeneration, treatment of vulvogaging atrophy and others.

Turning now to specific uses of the system 10, the invention also provides use of the colposuction system 10 to mediate effects associated with aging. Among the regions of the body that are affected during aging include the vagina and external genitalia. As estrogen levels drop over time the internal vaginal walls lose strength and thickness. But it's not just aging that can take its toll. The stress of childbirth can also affect sensation in the region as the vaginal walls undergo some significant pressure—not to mention the hormonal changes. Often these changes can affect the subject's sex life, with sex becoming less enjoyable or even painful.

Accordingly, the invention provides methods for improving sexual sensation to the user and/or to a sexual partner by way of use of the colposuction system 10. In particular, by using the system 10 to generate negative pressure against the vaginal walls, the method increases vaginal blood flow. This increased blood flow causes edema of the vaginal walls and/or hypertrophy of the vaginal wall muscle. Temporarily, this increases vaginal tightness and increases sensitivity, and reduces dryness, which can result in increased enjoyment for the subject and/or the subject's partner.

Continued use of the colposuction system 10 can extend the above effects for extended periods of time and can result in increased muscle thickness and tone. Thus, the invention also provides a method for affecting vaginal muscle tissue in a subject, which includes applying a sufficient amount of negative pressure to a subject's vaginal walls using the colposuction system 10 and for a sufficient time to cause edema of the vaginal tissue; and periodically repeating the application.

While creating edema does provide some benefit, significantly better results are achieved when using the colposuction system 10 to form micro-traumas, which stimulate vaginal tissue repair, tissue regeneration and rejuvenation. An exemplary method of inducing vaginal tissue regeneration, includes providing a patient in need of vaginal tissue regeneration; inserting the body 12 of the colposuction system 10 into the vagina of the patient; applying negative pressure within the chamber 20 so that the body 12 contacts the vaginal tissue along a length of the vaginal wall to be regenerated; maintaining sufficient negative pressure over a sufficient time or repeatedly applying sufficient negative pressure to cause micro-trauma of the vaginal tissue along the length of the vaginal wall to be regenerated; and releasing the negative pressure and removing the body 12 from the vagina. By inducing micro-traumas, the system 10 activates reparative cells and nutrients in the area of the injured vaginal tissue. This is achieved using more extreme pressure differences (e.g. 1 mmHg to 180 mmHg) against the vaginal tissue.

In view of the above, the invention also provides use of the system 10 to treat or ameliorate the effects of vaginal atrophy. Further, continued use of the system 10 can treat or mediate symptoms associated with pelvic floor disorders. In particular, the invention provides use of the system 10 to treat or ameliorate urinary incontinence (leaking of urine) and pelvic organ prolapse (weakening of the muscles and tissues supporting the organs in the pelvis). The conditions within this paragraph are preferably treated by way of creating micro-traumas within the vaginal tissue using the system at negative pressures from about 1 mmHg to about 180 mmHg. Alternatively, or in addition, they may be treated by pretreating the subject's vaginal tissue for a vaginal dermal filling or vaginal filling with fat procedure; and performing a dermal filling or fat filling procedure on the pretreated tissue.

The colposuction system 10 can also be used in various vaginal dermal filling or vaginal fat filling procedures to create tissues more accepting of a greater volume of dermal fillers or fat. Vaginal dermal filling or fat filling can be used as a means of improving how well tissues ‘feel’ and work. While dermal fillers are usually associated with face and lip enhancement, a carefully injected measure of filler in the right places can help restore the genital area making it feel healthier and stronger. Among the more popular dermal filling treatments include those containing hyaluronic acid gel. Hyaluronic acid is a naturally occurring substance found in cells and tissue fluids, which acts to maintain water content in the tissue and thus makes tissue more plump and pliable, which tends to decrease with age. In particular, the methods of the invention provide a pretreatment for vaginal dermal filling and vaginal fat filling procedures by way of generating regions along the vaginal walls that increase the acceptance of dermal fillers and/or fat fillers. Accordingly, a patient in need of dermal filling or fat filling can be pretreated using the system 10 for more effective loading of dermal fillers and/or fat fillers.

In an exemplary embodiment, a method of pretreating vaginal tissue for vaginal dermal filling or vaginal filling with fat is provided, which includes: providing a patient in need of a vaginal dermal filling or a vaginal filling with fat procedure; inserting the body 12 of the colposuction system 10 into the vagina of the patient; applying negative pressure within the chamber 20 so that the body 12 contacts the vaginal tissue along a length of the vaginal wall that is to be treated with the dermal filling or fat filling procedure; maintaining the negative pressure over time to cause edema of the vaginal tissue along the length of the vaginal wall to be treated or repeatedly applying negative pressure to cause the same; releasing the negative pressure and removing the body 12 from the vagina; and designating the swollen vaginal tissue as a pretreated region for the vaginal dermal filling or vaginal fat filling procedure while the pretreated region remains swollen. Afterwards, the swollen region is treated by way of a vaginal dermal tissue filling procedure or vaginal filling with fat procedure. In some embodiments, the maintained negative pressure or repeatedly applied negative pressure is from 1 mmHg to 180 mmHg.

As the disclosure provides a pretreatment for dermal filling or fat filling procedures, one of ordinary skill in the art will appreciate the appropriateness of potential dermal filling and fat filling procedures that can be performed following pretreatment.

Again, while the invention has been described in detail in connection with only a limited number of embodiments, it should be readily understood that the invention is not limited to such disclosed embodiments. Rather, the invention can be modified to incorporate any number of variations, alterations, substitutions, or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A colposuction system for applying negative pressure against walls of a vaginal canal, the system comprising:
a. an elongated body comprising an inner chamber that slidably houses a piston block, wherein the piston block is sealed air-tight against the inner chamber;
b. a plurality of channels that fluidly couple the inner chamber to an outer surface of the body, wherein the plurality of channels is positioned distal to the piston block; and
c. means for pulling the piston block proximally thereby applying negative pressure through the plurality of channels.

2. The colposuction system of claim 1, wherein the body comprises a rigid hollow core surrounded by a deformable or compressible coating layer.

3. The colposuction system of claim 1, wherein the body is deformable or compressible for contacting and following a contour of a vaginal canal when sufficient negative pressure is applied.

4. The colposuction system of claim 3, wherein the body is configured to deform so that at least 50% of a body surface area, defined by a region encompassing the plurality of channels, contacts vaginal tissue when the negative pressure is applied.

5. The colposuction system of claim 3, wherein the body is configured to deform so that at least 75% of a body surface area, defined by a region encompassing the plurality of channels, contacts vaginal tissue when the negative pressure is applied, optionally between 75% and 100% of the body surface area, defined by a region encompassing the plurality of channels, contacts vaginal tissue when the negative pressure is applied.

6. The colposuction system of claim 1, wherein the means for pulling the piston block proximally is selected from the group consisting of an automated pump, a squeeze bulb pump, and a pull line coupled to the piston block.

7. The colposuction system of claim 1, further comprising embedded LED lighting.

8. A method of pretreating vaginal tissue for vaginal dermal filling or vaginal filling with fat, the method comprising:
a. providing a patient for a vaginal dermal filling or a vaginal filling with fat procedure;
b. inserting the body of the system of claim 1 into the vagina of the patient;
c. applying negative pressure within the chamber so that the body contacts the vaginal tissue along a length of the vaginal wall that is to be treated with the dermal filling or fat filling procedure;
d. maintaining sufficient negative pressure over time or repeatedly applying sufficient negative pressure to cause edema and hypertrophy of the vaginal tissue along the length of the vaginal wall to be treated;
e. releasing the negative pressure and removing the body from the vagina; and
f. designating the swollen length of vaginal tissue as a pretreated region for the vaginal dermal filling or vaginal fat filling procedure while the pretreated region remains swollen.

9. The method of claim 8, wherein the maintained negative pressure or repeatedly applied negative pressure is from 1 mmHg to 180 mmHg.

10. A method of inducing vaginal tissue regeneration, the method comprising:
a. providing a patient in need of vaginal tissue regeneration;
b. inserting the body of the system of claim 1 into the vagina of the patient;
c. applying negative pressure within the chamber so that the body contacts the vaginal tissue along a length of the vaginal wall to be regenerated;
d. maintaining sufficient negative pressure over a sufficient time or repeatedly applying sufficient negative pressure to cause micro-trauma of the vaginal tissue along the length of the vaginal wall to be regenerated; and
e. releasing the negative pressure and removing the body from the vagina.

11. A method of causing edema of vaginal tissue, the method comprising applying a sufficient amount of negative pressure to a subject's vaginal tissue with the colposuction system according to claim 1 and for a sufficient time to cause edema of the vaginal tissue.

12. A method of improving sexual sensation in a subject or a partner, the method comprising providing a subject in need of improving sexual sensation or having a partner in need of improving sexual sensation; and performing the method of claim 11 on the subject.

13. The method of claim 12, wherein the negative pressure is applied to an area designated as a G-spot in the subject.

14. A method for affecting vaginal muscle tissue in a subject, the method comprising applying a sufficient amount of negative pressure to a subject's vaginal walls with the colposuction system according to claim 1 and for a sufficient time to cause edema of the vaginal tissue; and periodically repeating the application.

15. A method of treating or ameliorating urinary incontinence in a subject, the method comprising providing a subject suffering from urinary incontinence; performing the method of claim 8 on the subject; and performing a dermal filling or fat filling procedure on the pretreated tissue.

16. A method of treating or ameliorating urinary incontinence in a subject, the method comprising providing a subject suffering from urinary incontinence; and performing the method of claim 10 on the subject.

17. A method of treating or ameliorating urinary incontinence in a subject, the method comprising providing a subject suffering from urinary incontinence; and performing the method of claim 14 on the subject until urinary incontinence is reduced or prevented.

18. A method of preventing or ameliorating pelvic organ prolapse in a subject, the method comprising providing a subject suffering from or at risk of suffering from pelvic organ prolapse; performing the method of claim 8 on the subject; and performing a dermal filling or fat filling procedure on the pretreated tissue.

19. A method of preventing or ameliorating pelvic organ prolapse in a subject, the method comprising providing a subject suffering from or at risk of suffering from pelvic organ prolapse; and performing the method of claim 10 on the subject.

20. A method for progressive dilation of a stenoted vaginal canal, the method comprising providing a subject suffering from a stenoted vaginal canal; and periodically performing the method of claim 14 on the subject.
